(19) United States
(12) Patent Application Publication
(10) Pub. No.: US 2025/0360063 A1
(43) Pub. Date: Nov. 27, 2025

(54) WATER-IN-OIL EMULSIFIED SOLID COSMETIC

(71) Applicant: SHISEIDO COMPANY, LTD., Tokyo (JP)

(72) Inventors: Megumi ANDO, Tokyo (JP); Angela QUACH, Tokyo (JP)

(21) Appl. No.: 18/874,772

(22) PCT Filed: Jun. 30, 2023
(86) PCT No.: PCT/JP2023/024345

§ 371 Date: Dec. 13, 2024

(30) Foreign Application Priority Data
Jul. 14, 2022 (JP) 2022-113474

(51) Int. Cl. A61K 8/06; A61K 8/02; A61K 8/92; A61Q 1/06

(52) U.S. Cl. CPC A61K 8/064; A61K 8/0229; A61K 8/92; A61Q 1/06

(57) ABSTRACT

One aspect of the present invention is a water-in-oil emulsified solid cosmetic including: an internal aqueous phase in which powder is dispersed in an aqueous medium; and an external oil phase thickened or solidified with wax, wherein an average primary particle size of the powder is 1.0 μm or more.

### TECHNICAL FIELD

The present invention relates to a water-in-oil emulsified solid cosmetic.

### BACKGROUND OF THE INVENTION

Solid cosmetic is a dosage form used for various items such as skincare cosmetics and makeup cosmetics. For example, in makeup cosmetics, consumers tend to demand a variety of qualities in terms of color and texture. In recent years, as for the finished texture of makeup cosmetics when applied to the skin, a matte feeling in which gloss is reduced and the skin looks flawless has been demanded. In order to achieve a matte finish, studies have generally been made to incorporate various powders into solid cosmetics.

For example, Patent Document 1 discloses an oil-based solid cosmetic formulated with low-melting point wax, plate powder, and spherical powder. The oil-based solid cosmetic of Patent Document 1 is formulated with a specific amount of these ingredients to obtain a natural matte feeling.

### RELATED-ART DOCUMENT

### Patent Document

Patent Document 1: Japanese Unexamined Patent Application Publication No. 2000-119134

### SUMMARY OF THE INVENTION

### Problems to be Solved by the Invention

However, the conventional solid cosmetic containing powders has a problem of poor spreading upon application, causing a user to feel dry, although a matte finish can be achieved after application to the skin.

An object of one aspect of the present invention is to provide a water-in-oil emulsified solid cosmetic that spreads smoothly when applied to the skin, gives a fresh feel to a user, and achieves a matte finish after application.

### Means for Solving the Problems

In one aspect of the present invention, a water-in-oil emulsified solid cosmetic includes: an internal aqueous phase in which powder is dispersed in an aqueous medium; and an external oil phase thickened or solidified with wax, wherein an average primary particle size of the powder is 1.0 μm or more.

### Effects of the Invention

According to one aspect of the present invention, a water-in-oil emulsified solid cosmetic that spreads smoothly when applied to the skin, gives a fresh feel to a user, and achieves a matte finish after application, can be provided.

### DETAILED DESCRIPTION OF THE INVENTION

Embodiments of the present invention will be described in detail below. Embodiments of the present invention are not limited by the following description, and may be modified as needed without departing from the scope of the present invention. In the present specification, “X to Y” indicating a numerical range is meant to include a lower limit and an upper limit that are given as numerical values before and after “to”, unless otherwise noted.

A water-in-oil emulsified solid cosmetic according to the present embodiment contains an internal aqueous phase in which powder is dispersed in an aqueous medium and an external oil phase thickened or solidified with wax. The term “solid” as used in the present specification is understood to mean what is commonly used in the field of cosmetics, and can be defined as, for example, a form or state in which the entire composition does not exhibit fluidity at a temperature of 50° C. or lower and does not exhibit significant deformation under normal storage conditions.

Each component included in the water-in-oil emulsified solid cosmetic according to the present invention is described below in detail.

The powder means powder that can be used normally in cosmetics and pharmaceuticals and surface-hydrophilic powder that can be stably dispersed in an aqueous system. The surface-hydrophilic powder includes hydrophilic powder that has not been surface-hydrophobized and hydrophobic or hydrophilic powder that has been surface-treated with a hydrophilic substance. In the present specification, the powder means powder contained in the internal aqueous phase.

The average primary particle size of the powder is 1.0 μm or more. The water-in-oil emulsified solid cosmetic according to the present embodiment is demulsified when applied to the skin, so that it spreads smoothly and gives a fresh feel to a user. After the water-in-oil emulsified solid cosmetic is applied to the skin, the water-in-oil emulsified solid cosmetic that has been demulsified forms an oil phase on the skin and an aqueous phase on the oil phase. Then, the water in the aqueous phase evaporates and the powder having an average primary particle size of 1.0 μm or more contained in the aqueous phase remains in the oil phase covering the skin, so that the water-in-oil emulsified solid cosmetic can achieve a matte finish.

As used herein, the average primary particle size means the average of primary particle sizes measured by a commonly used method, and is specifically determined as the arithmetic average of the major and minor axes of particles from transmission electron micrographs.

The average primary particle size of the powder is preferably 1.0 μm to 50.0 μm, more preferably 1.5 μm to 13 pm. When the average primary particle size of the powder is 1.0 μm to 50 μm, the water-in-oil emulsified solid cosmetic spreads more smoothly when applied to the skin, and gives a fresher feel to a user. When the average primary particle size of the powder is 1.5 μm to 13 μm, the water-in-oil emulsified solid cosmetic spreads further more smoothly when applied to the skin, and gives a further fresher feel to a user, and achieves a more matte finish after application.

The content of the powder is preferably 1 mass % to 25 mass %, more preferably 1 mass % to 20 mass %, and further more preferably 5 mass % to 15 mass % with respect to the water-in-oil emulsified solid cosmetic. When the content of the powder is 1 mass % to 25 mass % with respect to the water-in-oil emulsified solid cosmetic, the water-in-oil emulsified solid cosmetic spreads more smoothly when applied to the skin, and gives a fresher feel to a user, and achieves a more matte finish after application.

The shape of the powder may be plate or spherical. Examples of the powder include inorganic powders such as talc, kaolin, sericite, mica (white mica, synthetic mica, triethoxysilylethyl polydimethylsiloxyethyl dimethicone-treated synthetic phlogopite, phlogopite, red mica, black mica, and lithia mica), vermiculite, magnesium carbonate, calcium carbonate, diatomaceous earth, magnesium silicate, calcium silicate, aluminum silicate, barium silicate, strontium silicate, calcium myristate, metal tungstate, silica, silicone, cellulose powder, hydroxyapatite, zeolite, boron nitride, and ceramic powder; and lauroyl lysine. Among these powders, one type may be used alone or two or more types may be used in combination.

Among these, the powder preferably contains one or more selected from mica, silica, silicone, talc, cellulose powder, and lauroyl lysine. Accordingly, the water-in-oil emulsified solid cosmetic can achieve a matte finish after application.

The powder is more preferably inorganic powder. Accordingly, the water-in-oil emulsified solid cosmetic can achieve an even more matte finish after application.

The aspect ratio of mica is preferably 10 to 80 and more preferably 20 to 60. When the mica aspect ratio is 10 to 80, the water-in-oil emulsified solid cosmetic can achieve an even more matte finish after application.

Examples of the silicone used for the powder include a diphenyl dimethicone/vinyl diphenyl dimethicone/silsesquioxane crosspolymer, a dimethicone/vinyl dimethicone crosspolymer, a vinyl dimethicone/methicone silsesquioxane crosspolymer, a diphenyl dimethicone/vinyl diphenyl dimethicone/silsesquioxane crosspolymer, and the like. Examples of commercially available products of the above crosspolymers include, KSP series (KSP-100, KSP-101, KSP-102, KSP-105, KSP-300, KSP-411, KSP-441 (manufactured by Shin-Etsu Chemical Co., Ltd.)), and the like.

The aqueous medium in which the powder is dispersed may be water or a mixture of water and other aqueous media such as lower alcohols. The content of the aqueous medium in the water-in-oil emulsified solid cosmetic is preferably 1 mass % to 70 mass %, more preferably 3 mass % to 60 mass %, and further more preferably 5 mass % to 50 mass %. When the content of the aqueous medium in the water-in-oil emulsified solid cosmetic is 1 mass % to 70 masst, the powder can be dispersed sufficiently.

In addition to the powder and the aqueous medium, other optional ingredients may be further incorporated into the internal aqueous phase. Examples of other optional ingredients include, but are not limited to, various water-soluble ingredients such as moisturizers, thickeners, dispersants, preservatives, agents, stabilizers, and the like.

Examples of the moisturizer include 1,3-butylene glycol, polyethylene glycol, propylene glycol, dipropylene glycol (abbreviated as DPG), hexylene glycol, glycerin, diglycerin, xylitol, maltol, maltose, D-mannite, trehalose, erythritol, propanediol, glucose, polyoxyethylene methyl glucoside, and the like. When the internal aqueous phase contains the moisturizer, it can provide a more moisturizing feeling to the user's skin while maintaining a matte finish after application.

Examples of the thickener includes plant-based polymers such as xanthan gum, cellulose gum, gum arabic, carrageenan, pectin, agar, pyrus cydonia seed (pyrus cydonia) extract, starch, algae colloid (extract of Phaeophyta), mucopolysaccharide, tremella fuciformis polysaccharide, and succinoglycan; microbial-based polymers such as dextran and pullulan; animal-based polymers such as collagen, casein, and gelatin; starch-based polymers such as carboxymethyl starch and methyl hydroxypropyl starch; alginic acid-based polymers such as sodium alginate; vinyl-based polymers such as carboxy vinyl polymer (carbomer); acrylic-based polymers such as polyoxyethylene-based polymers, polyoxyethylene polyoxypropylene copolymer-based polymers, sodium polyacrylate, and polyacrylamide; inorganic-based water-soluble polymers such as bentonite, aluminum magnesium silicate, and laponite; and the like.

The content of the thickener is preferably in the range of 0.005 mass % to 3 mass %, more preferably 0.05 mass % to 2.8 mass % with respect to the water-in-oil emulsified solid cosmetic. The lower limit of the content range may be 0.005 mass %, preferably 0.01 mass %, more preferably 0.05 mass %, and further more preferably 0.1 mass %. The upper limit combined with any of these lower limits may be 3 mass %, preferably 2.8 mass %.

The dispersant means a substance that can be adsorbed onto the surface of the powder and disperse the powder uniformly in the aqueous medium without agglomeration. The dispersant may be any aqueous dispersant that is commonly used in cosmetics and pharmaceuticals. Examples of the dispersant include fatty acid soaps, non-ionic surfactants, condensed phosphate compounds, polycarboxylate compounds (ammonium polycarboxylate and the like), amino acid compounds, polyacrylic acid compounds, amines (amino alcohols and the like), and the like.

Examples of the fatty acid soap include sodium laurate, sodium myristate, sodium palmitate, sodium stearate, potassium laurate, potassium myristate, potassium palmitate, potassium stearate, calcium stearate, and the like. Potassium hydroxide or sodium hydroxide may be incorporated first, and then fatty acids such as lauric acid, myristic acid, palmitic acid, and/or stearic acid may be added thereto to act as the fatty acid soap during the preparation of the aqueous phase.

Examples of the non-ionic surfactant includes POE-sorbitan fatty acid esters such as POE-sorbitan monooleate, POE-sorbitan monostearate, and POE-sorbitan tetraoleate; POE-sorbit fatty acid esters such as POE-sorbit monolaurate, POE-sorbit monooleate, POE-sorbit pentaoleate, and POE-sorbit monostearate; POE-glycerin fatty acid esters such as POE-monooleates including POE-glycerin monostearate, POE-glycerin monoisostearate, and POE-glycerin triisostearate; POE-fatty acid esters such as POE-distearate, POE-monodioleate, and ethylene glycol distearate; pluronic surfactants such as Pluronic (registered trademark); POE·POP-alkyl ethers such as POE·POP-cetyl ether, POE·POP-2 decyltetradecyl ether, POE·POP-monobutyl ether, POE·POP-hydrogenated lanolin, and POE·POP-glycerin ether; tetra POE·tetra-POP-ethylenediamine condensates such as tetronic; POE-castor oil hydrogenated castor oil derivatives such as POE-castor oil, POE-hydrogenated castor oil, POE-hydrogenated castor oil monoisostearate, POE-hydrogenated castor oil triisostearate, POE-hydrogenated castor oil monopyroglutamate monoisostearate diester, and POE-hydrogenated castor oil maleate; POE-beeswax/lanolin derivatives such as POE-sorbit beeswax; alkanol amides such as coconut fatty acid diethanol amide, lauric acid monoethanol amide, and fatty acid isopropanolamide; POE-propylene glycol fatty acid ester, POE-alkyl amine, POE-fatty acid amide, and alkylethoxydimethylamine oxide; hydrophilic surfactants in which hydrophile-lipophile balance (HLB) is 8 or more, preferably 10 or more, such as trioleyl phosphate; and the like.

Examples of the condensed phosphate compound include trisodium phosphate, sodium pyrophosphate, sodium hexametaphosphate, and the like.

Examples of the preservative include phenoxyethanol.

Examples of the agent include vitamins such as vitamin A, vitamin B3 (niacinamide), retinol, retinol palmitate, inositol, pyridoxine hydrochloride, benzyl nicotinate, nicotinamide, DL-α-tocopherol nicotinate, magnesium ascorbyl phosphate, ascorbic acid 2-glucoside, vitamin D2 (ergocalciferol), dl-α-tocopherol 2-L ascorbic acid phosphoric acid diester potassium salt, dl-α-tocopherol, dl-α-tocopherol acetate, pantothenic acid, and biotin; anti-inflammatory agents such as allantoin, azulene, and glycyrrhetinic acid; whitening agents such as arbutin, tranexamic acid, and potassium 4-methoxysalicylate; astringents such as zinc oxide and tannic acid; sulfur, lysozyme chloride, pyridoxine hydrochloride, γ-oryzanol, and the like.

The wax may be selected from waxes conventionally used in makeup cosmetics. Examples of the wax include solid oils and fats such as cocoa butter, coconut oil, horse oil, hardened coconut oil, palm oil, beef tallow, mutton tallow, hardened castor oil, and hydrogenated vegetable oil; hydrocarbons such as paraffin (straight-chain hydrocarbon), microcrystalline wax (branched-chain saturated hydrocarbon), ceresin wax, Japan wax, and Fischer-Tropsch wax; waxes such as beeswax, lanolin, carnauba wax, candelilla wax, rice bran wax, spermaceti wax, jojoba oil, bran wax, montan wax, kapok wax, bayberry wax, shellac wax, sugar cane wax, sunflower seed wax, isopropyl lanolate, hexyl laurate, hydrogenated lanolin, hard lanolin, POE lanolin alcohol ether, POE lanolin alcohol acetate, POE cholesterol ether, lanolin fatty acid polyethylene glycol, and POE hydrogenated lanolin alcohol ether; higher fatty acids such as myristic acid, palmitic acid, stearic acid, behenic acid, and hydroxystearic acid; and higher alcohols such as cetyl alcohol, stearyl alcohol, behenyl alcohol, myristyl alcohol, and cetostearyl alcohol. Among these waxes, one type may be used alone or two or more types may be used in combination. Among these, paraffin (straight-chain hydrocarbon), microcrystallin wax, rice bran wax, hydroxystearic acid, hydrogenated castor oil, and hydrogenated vegetable oil are preferred.

The content of the wax is preferably 0.1 mass % to 40 mass %, more preferably 1 mass % to 35 mass %, and further more preferably 2 mass % to 25 mass % with respect to the water-in-oil emulsified solid cosmetic. When the content of the wax is 0.1 mass % to 40 mass % with respect to the water-in-oil emulsified solid cosmetic, the water-in-oil emulsified solid cosmetic can be thickened or solidified to an appropriate consistency and can exhibit good usability.

The external oil phase of the water-in-oil emulsified solid cosmetic may be thickened or solidified with the wax and a gelling agent. Examples of the gelling agent include an oil-based gelling agent such as organic modified clay mineral, 12-hydroxystearic acid, starch fatty acid ester, dimethyl polysiloxane polymer, dextrin fatty acid ester, and the like. Among these gelling agents, one type may be used alone or two or more types may be used in combination.

The total content of the wax and the gelling agent is preferably 0.1 mass % to 40 mass %, more preferably 1 mass % to 35 mass %, and further more preferably 2 mass % to 25 mass % with respect to the water-in-oil emulsified solid cosmetic. When the total content of the wax and the gelling agent is 0.1 mass % to 40 mass % with respect to the water-in-oil emulsified solid cosmetic, the water-in-oil emulsified solid cosmetic can be thickened or solidified to an appropriate consistency and can exhibit good usability.

The external oil phase of the water-in-oil emulsified solid cosmetic contains the wax or liquid oil that is thickened or solidified by the wax and the gelling agent. Here, the liquid oil means oil that is liquid at room temperature (25° C.) and can be normally used in cosmetics and pharmaceuticals.

Examples of the liquid oil includes liquid oils and fats such as avocado oil, camellia oil, macadamia seed oil, mink oil, olive oil, castor oil, jojoba oil, triglyceride, and glyceryl trioctanoate; hydrocarbons such as liquid paraffin, squalane, paraffin, ceresin, and squalene; higher fatty acids such as lauric acid, myristic acid, palmitic acid, stearic acid, behenic acid, oleic acid, isostearic acid, linoleic acid, and linolenic acid; higher alcohols such as lauryl alcohol, cetyl alcohol, stearyl alcohol, behenyl alcohol, oleyl alcohol, monostearyl glycerol ether, monopalmityl glycerol ether, cholesterol, phytosterol, and isostearyl alcohol; ester oils such as caprylic/capric triglyceride, pentaerythrityl tetraethylhexanoate, isononyl isononanoate, isopropyl myristate, cetyl octanoate, octyldodecyl myristate, butyl stearate, decyl oleate, ethylene glycol dioctanoate, diisostearyl malate, trimethylolpropane trioctanoate, trimethylolpropane triisostearate, glyceryl trioctanoate, glyceryl triisostearate, pentaerythritol tetraoctanoate, ethyl acetate, butyl acetate, and amyl acetate; straight-chain silicone oils such as dimethyl polysiloxane, methyl polysiloxane (dimethicone), methyl phenyl polysiloxane, and methyl hydrogen polysiloxane; cyclic silicone oils such as decamethylcyclopentasiloxane, dodecamethylcyclohexasiloxane, and octamethylcyclotetrasiloxane; and the like. Among these liquid oils, one type may be used alone or two or more types may be used in combination. Among these, straight-chain silicone oils such as dimethyl polysiloxane, dodecamethylpentasiloxane, decamethyltetrasiloxane, and pentaerythritol tetraoctanoate; and cyclic silicone oils such as decamethylcyclopentasiloxane, are preferably blended as a main component of the liquid oil.

The external oil phase preferably contains 15 mass % or more of residual oil with respect to the water-in-oil emulsified solid cosmetic. Here, the residual oil means non-volatile liquid oil among the liquid oil. Examples of the non-volatile liquid oil include caprylic/capric triglyceride, pentaerythrityl tetraethylhexanoate, and the like. Examples of the volatile liquid oil include dimethicone. When the external oil phase contains 15 mass % or more of the residual oil with respect to the water-in-oil emulsified solid cosmetic, the water-in-oil emulsified solid cosmetic can give a moisturizing feeling to the user's skin while achieving a matte finish after application. In particular, when the water-in-oil emulsified solid cosmetic is a lipstick, the external oil phase preferably contains 15 mass % or more of the residual oil with respect to the water-in-oil emulsified solid cosmetic.

The external oil phase preferably contains a color material. Examples of the color material include inorganic white pigments (titanium dioxide and zinc oxide); inorganic red pigments (iron oxide (Bengala) and iron titanate); inorganic brown pigments (γ-iron oxide); inorganic yellow pigments (yellow iron oxide and ochre); inorganic black pigments (black iron oxide, carbon, and lower titanium oxide); inorganic purple pigments (mango violet and cobalt violet); inorganic green pigments (chromium oxide, chromium hydroxide, and cobalt titanate); inorganic blue pigments (ultramarine blue and prussian blue); pearl pigments (titanium oxide-coated mica, titanium oxide-coated bismuth oxychloride, titanium oxide-coated talc, colored titanium oxide-coated mica, bismuth oxychloride, fish scale foil); metallic powder pigments (aluminum powder and copper powder); organic pigments (Red No. 202, Red No. 205, Red No. 220, Red No. 228, Red No. 405, Orange No. 203, Orange No. 204, Yellow No. 205, Yellow No. 401, Blue No. 404); organic pigments of zirconium, barium, and aluminum lakes (Red No. 3, Red No. 104, Red No. 227, Red No. 401, Orange No. 205, Yellow No. 4, Yellow No. 202, Green No. 3, Blue No. 1); natural pigments (chlorophyll, carotenoids (β-carotene), carthamin, cochineal, chalcone, curcumin, betanin, flavonol, flavone, anthocyanidin, anthraquinone, and naphthoquinone); functional pigments (boron nitride, photochromic pigment, synthetic fluorophlogopite, iron-containing synthetic fluorophlogopite, fine particle composite powder (hybrid fine powder)); oil-soluble dyes; and the like. Among these color materials, one type may be used alone or two or more types may be used in combination.

Among these, the organic pigments or the oil-soluble dyes are preferable as the color material. The organic pigments or the oil-soluble dyes are preferably contained in the external oil phase. When the external oil phase contains the organic pigments or the oil-soluble dyes, the water-in-oil emulsified solid cosmetic can stably exhibit bright color development while maintaining a matte finish after application to the skin.

The content of color material is preferably 0.005 mass % to 30 mass %, more preferably 0.01 mass % to 25 mass %, and further more preferably 0.01 mass % to 15 mass % with respect to the water-in-oil emulsified solid cosmetic. When the content of the organic pigment is 0.005 mass % to 30 mass % with respect to the water-in-oil emulsified solid cosmetic, the water-in-oil emulsified solid cosmetic spreads more smoothly when applied to the skin, and gives a fresher feel to a user.

The external oil phase may contain 15 mass or less of semisolid oil with respect to the water-in-oil emulsified solid cosmetic. More preferably, the content of the semisolid oil is 0.1 mass % to 20 mass % with respect to the water-in-oil emulsified solid cosmetic. The semisolid oil means oil that has a melting point above room temperature (25° C.), does not solidify completely at room temperature, and is distinct from liquid oil and solid oil. The semisolid oil does not include the wax or the gelling agent. Examples of the semisolid oil include pentaerythritol fatty acid esters such as pentaerythrityl tetrabehenate/benzoate/ethylhexanoate, dipentaerythrityl hexahydroxystearate, and dipentaerythrityl tetrahydroxystearate/tetraisostearate; phytosterol fatty acid esters such as macadamia seed oil fatty acid phytosteryl (macadamia seed oil polyglyceryl-6 esters behenate, and the like); dimer dilinoleic acid esters such as phytosteryl/behenyl dimer dilinoleate; lanolin; vegetable oils such as butyrospermum parkii (shea) butter, petrolatum, partially hydrogenated coconut oil; partially hydrogenated jojoba oil, bis-diglyceryl polyacyladipate-2, and the like. When the external oil phase contains 15 mass % or less of the semisolid oil with respect to the water-in-oil emulsified solid cosmetic, the water-in-oil emulsified solid cosmetic can provide a moisturizing feeling to the user's skin while maintaining a matte finish after application. The water-in-oil emulsified solid cosmetic need not necessarily contain the semisolid oil.

The external oil phase may contain 20 mass % or less of a film-forming agent with respect to the water-in-oil emulsified solid cosmetic. The film-forming agent is not particularly limited as long as it is normally used in cosmetics. Examples of the film-forming agent include trimethylsiloxysilicate, dimethylaminomethacrylate quaternized salt, vinylpyrrolidone methacrylate-N,N-dimethyl-ethylantinioethyl salt copolymer, silicone/polyether-based polyurethane resin, (methacryloyloxyethyl carboxybetaine/methacrylalkyl) copolymer, dextrin, (vinylpyrrolidone/VA) copolymer, ammonium acrylates copolymer, polyvinyl alcohol, ethyl polyacrylate, (acrylates/octylacrylamide) copolymer, (acrylates/propyl trimethicone methacrylate) copolymer, polyvinyl acetate, (acrylates/dimethicone) copolymer, 3-[tris(trimethylsiloxane)silyl]propyl carbamate pullulan, polyether graft silicone acrylates, fluoro-modified silicone resin, and the like. When the external oil phase contains 20 mass % or less of the film-forming agent with respect to the water-in-oil emulsified solid cosmetic, the water-in-oil emulsified solid cosmetic can provide a moisturizing feeling to the user's skin while maintaining a matte finish after application. The water-in-oil emulsified solid cosmetic need not necessarily contain the film-forming agent.

The water-in-oil emulsified solid cosmetic agent may contain a surfactant used to emulsify the internal aqueous phase into the external oil phase. As the surfactant, emulsifiers that can generally be used in water-in-oil emulsified compositions, that is, lipophilic emulsifiers (HLB=7 or less) can be used. Specifically, examples of the surfactant include sorbitan fatty acid esters such as sorbitan monolaurate, sorbitan monooleate, sorbitan monoisostearate, sorbitan tristearate, and sorbitan sesquiisostearate; glycerol fatty acid esters such as glycerol monostearate, glycerol monoolate, glycerol isostearate, diglycerol diisostearate, and decaglyceryl pentaisostearate; polyoxyethylene hydrogenated castor oil such as POE (5), POE (7.5), POE (10) hydrogenated castor oil, and PEG-60 hydrogenated castor oil; high molecular weight lipophilic activators such as dipolyhydroxystearate esters, polyglyceryl dipolyhydroxystearate-2 (PGPH, manufactured by Cognis), and PEG-30 dipolyhydroxystearate (Arlacel P135, manufactured by Uniqema); polyether-based silicone such as polyether-modified silicones such as cetyl dimethicone copolyol (for example, ABIL EM90, manufactured by Goldschmidt), cetyl PEG/PPG-10/1 dimethicone, and PEG-10 dimethicone (for example, KSG series manufactured by Shin-Etsu Chemical Co., Ltd.), and crosslinked polyether-modified silicones (for example, KSG series manufactured by Shin-Etsu Chemical Co., Ltd.); and polyglycerin-based silicones such as polyglycerin-modified silicones and alkyl co-modified polyglycerin-modified silicones; and the like.

In the water-in-oil emulsified solid cosmetic, in addition to the above-mentioned ingredients, other optional ingredients that can be incorporated into the cosmetic can be incorporated into the internal aqueous phase or the external oil phase to the extent that the effect of the present embodiment is not impaired.

The method of producing the water-in-oil emulsified solid cosmetic may be a method according to the commonly used method. The outline of the method will be described below.
(1) An aqueous medium and other optional components are mixed while heating as necessary, and then powder is added and stirred and mixed with a homomixer or the like to prepare an aqueous phase component.
(2) Wax and other optional components are mixed while heating as necessary to prepare an oil phase component.
(3) An emulsifier is optionally added to the oil phase component, and the aqueous phase component is added thereto and stirred and mixed with a homomixer or the like to obtain an emulsion.
(4) The obtained emulsion is filled in a container and cooled and solidified to obtain the water-in-oil emulsified solid cosmetic of the present embodiment.

The water-in-oil emulsified solid cosmetic according to the present embodiment may be a makeup cosmetic such as lipstick, lip balm, foundation, eye shadow, eyeliner, mascara, blush, and concealer, skin care cosmetic, makeup base, hair cosmetic, and the like. Among these, the water-in-oil emulsified solid cosmetic has a remarkable effect when it is a makeup cosmetic, and is preferable when it is a lipstick because it spreads smoothly when applied to the skin, gives a fresh feel to a user, and achieves a matte finish after application. The shape of the water-in-oil emulsified solid cosmetic according to the present embodiment is not particularly limited, but may be a balm filled in a wide-mouth container or a stick.

### EXAMPLES

Embodiments will be described in more detail below with Examples and Comparative Examples, but the embodiments are not limited by these Examples and Comparative Examples.

Water-in-oil emulsified solid cosmetics were prepared with the compositions presented in Tables 1 to 4 by the following methods, and each obtained water-in-oil emulsified solid cosmetic was evaluated by the following methods. Details of the main components presented in Tables 1 to 4 are as follows. In the present specification and in the tables, particle size means the average primary particle size.
Synthetic phlogopite (particle size 2.4 to 2.9 μm) (Mica STY, manufactured by Topy Industries Ltd.)
Synthetic phlogopite (particle size 12.5 μm) (Mica ST (HS), manufactured by Topy Industries Ltd.).
Triethoxysilylethyl polydimethylsiloxyethyl dimethicone-treated synthetic phlogopite (particle size 11.3 μm) (SWD-Mica STY, manufactured by Topy Industries Ltd.)
Silica (particle size 0.55 μm) (COSMO 55, manufactured by JGC Catalysts and Chemicals Ltd.):
Silica (particle size 1 μm) (TMS-01, manufactured by Tayca Co., Ltd.)
Silica (particle size 5 μm) (SATINIER M5, manufactured by JGC Catalysts and Chemicals Ltd.)
Calcium myristate (particle size 7 to 11 μm) (Mirikaru MB, manufactured by NOF Corporation)
Cellulose (particle size 5 to 25 μm) (Celluflow C-25, manufactured by Chisso Corporation)
Dimethicone (silicone KF-96A-1.5CS)
Caprylic/capric triglyceride (O.D.O)
Distearyl malate (Cosmol (registered trademark) 222, manufactured by Nisshin Oillio Group, Ltd.)
Pentaerythrityl tetraethylhexanoate (RA-PE-408, manufactured by Nippon Fine Chemical Co., Ltd.)
Paraffin (Paramix 91): Glycerin (DYNAMITE GLYCERINE, manufactured by NOF Corporation)
DPG (dipropylene glycol, ADEKA Corporation)
Xanthan gum (Keltrol (registered trademark), manufactured by Kelco Inc.)
Silica dimethyl silylate (Aerosil (registered trademark) R972)
Cetyl PEG/PPG-10/1 dimethicone (Silicone KF-6048)
PEG-60 hydrogenated castor oil (Nikkol (registered trademark) HCO-60)
Calcium stearate (calcium stearate, manufactured by Tannan Kagaku Kogyo Co., Ltd.)
Niacinamide (Silicone KF-96A-1.5CS)
Macadamia seed oil polyglyceryl-6 esters behenate (S Face VL-211)
Trimethylsiloxysilicate (component A of SSD-R2-ID)
Isodododecane (component B of SSD-R2-ID)

### <Preparation of Water-in-Oil Emulsified Solid Cosmetic>

Each component with the composition presented in Tables 1 to 4 was put into a beaker and mixed at room temperature (25° C.) to obtain water-in-oil emulsified solid cosmetics of Examples 1 to 18 and Comparative Examples 1 and 2.

### <Evaluation>

The water-in-oil emulsified solid cosmetics of Examples 1 to 18 and Comparative Examples 1 and 2 were respectively applied to the lips of five people on an expert panel, and each person evaluated the comfort in application, matte feeling after application, and moisturizing feeling, based on the following evaluation criteria. Here, the comfort in application means an overall evaluation item of smoothness and freshness upon application. Then, an average score of the evaluation results of the five people was calculated based on the following evaluation criteria, with 4 points for a, 3 points for b, 2 points for c, and 1 point for d. A score of C or higher was considered acceptable. The evaluation results are presented in Tables 1 to 4.

### [Evaluation Criteria for Matte Feeling]

a: Very matte.
b: Matte.
c: Somewhat matte.
d: Glossy.

### [Evaluation Criteria for Comfort in Application]

a: Very comfortable to apply.
b: Comfortable to apply.
c: Somewhat comfortable to apply.
d: Not comfortable to apply.

### [Evaluation Criteria for Moisturizing Feeling]

a: Very moisturizing.
b: Moisturizing.
c: Somewhat moisturizing.
d: Not moisturizing (dry).

### [Evaluation Criteria for Average Score]

A: Average score of 3.5 or more points
B: Average score of 2.5 or more points and less than 3.5 points
C: Average score of 1.5 or more points and less than 2.5 points
D: Average score of less than 1.5 points

TABLE 1

| | | EXAM- | EXAM- | EXAM- | EXAM- | EXAM- | EXAM- |
|---|---|---|---|---|---|---|---|
| | | PLE | PLE | PLE | PLE | PLE | PLE |
| | | 1 | 2 | 3 | 4 | 5 | 6 |
| WATER | WATER | 18.05 | 18.05 | 18.05 | 18.05 | 18.05 | 18.05 |
| POWDER | SYNTHETIC PHLOGOPITE | 10.00 | 5.00 | — | — | — | — |
| | (PARTICLE SIZE 2.4-2.9 μm) | | | | | | |
| | SYNTHETIC PHLOGOPITE | — | — | 5.00 | — | — | — |
| | (PARTICLE SIZE 12.5 μm) | | | | | | |
| | TRIETHOXYSILYLETHYL | — | — | — | 5.00 | — | — |
| | POLYDIMETHYLSILOXYETHYL | | | | | | |
| | DIMETHICONE-TREATED | | | | | | |
| | SYNTHETIC PHLOGOPITE | | | | | | |
| | (PARTICLE SIZE 11.3 μm) | | | | | | |
| | SILICA (PARTICLE SIZE 0.55 μm) | — | — | — | — | — | — |
| | SILICA (PARTICLE SIZE 1 μm) | — | — | — | — | 10.00 | — |
| | SILICA (PARTICLE SIZE 5 μm) | — | — | — | — | — | 10.00 |
| | CALCIUM MYRISTATE | — | — | — | — | — | — |
| | (PARTICLE SIZE 7-11 μm) | | | | | | |
| | CELLULOSE | — | — | — | — | — | — |
| | (PARTICLE SIZE 5-25 μm) | | | | | | |
| LIQUID OIL | DIMETHICONE | 10.00 | — | — | — | 10.00 | 10.00 |
| | CAPRYLIC/CAPRIC | 22.45 | 11.45 | 11.45 | 11.45 | 22.45 | 22.45 |
| | TRIGLYCERIDE | | | | | | |
| | DISTEARYL MALATE | — | 10.00 | 10.00 | 10.00 | — | — |
| | PENTAERYTHRITYL | 8.00 | 13.00 | 13.00 | 13.00 | 8.00 | 8.00 |
| | TETRAETHYLHEXANOATE | | | | | | |
| WAX | PARAFFIN | 10.20 | 10.20 | 10.20 | 10.20 | 10.20 | 10.20 |
| | MICROCRYSTALLINE WAX | 0.80 | 0.80 | 0.80 | 0.80 | 0.80 | 0.80 |
| COLOR MATERIAL | COLOR MATERIAL | 5.00 | 10.00 | 10.00 | 10.00 | 5.00 | 5.00 |
| MOISTURIZER | GLYCERIN | 4.00 | 4.00 | 4.00 | 4.00 | 4.00 | 4.00 |
| | DPG | 6.00 | 6.00 | 6.00 | 6.00 | 6.00 | 6.00 |
| THICKENER | XANTHAN GUM | 0.15 | 0.15 | 0.15 | 0.15 | 0.15 | 0.15 |
| VISCOSITY | SILICA DIMETHYL SILYLATE | — | 1.00 | 1.00 | 1.00 | — | — |
| MODIFIER | | | | | | | |
| SURFACTANT | CETYL PEG/PPG-10/ | 4.00 | 4.00 | 4.00 | 4.00 | 4.00 | 4.00 |
| | 1 DIMETHICONE | | | | | | |
| | PEG-60 HYDROGENATED CASTOR OIL | 0.80 | 0.80 | 0.80 | 0.80 | 0.80 | 0.80 |
| STABILIZER | CALCIUM STEARATE | 0.05 | 0.05 | 0.05 | 0.05 | 0.05 | 0.05 |
| PRESERVATIVE | PHENOXYETHANOL | 0.50 | 0.50 | 0.50 | 0.50 | 0.50 | 0.50 |
| AGENT | NIACINAMIDE | — | 5.00 | 5.00 | 5.00 | — | — |
| TOTAL (MASS %) | | 100 | 100 | 100 | 100 | 100 | 100 |
| EVALUATION | MATTE FEELING | B | B | B | B | C | B |
| | COMFORT IN APPLICATION | A | A | A | A | A | A |
| | MOISTURIZING FEELING | A | A | A | A | A | A |

TABLE 2

| | | | | COMPAR- | COMPAR- | | |
|---|---|---|---|---|---|---|---|
| | | EXAM- | EXAM- | ATIVE | ATIVE | EXAM- | EXAM- |
| | | PLE | PLE | EXAM- | EXAM- | PLE | PLE |
| | | 7 | 8 | PLE 1 | PLE 2 | 9 | 10 |
| WATER | WATER | 18.05 | 18.05 | 18.05 | 18.05 | 23.05 | 12.05 |
| POWDER | SYNTHETIC PHLOGOPITE | — | — | — | — | 5.00 | 16.00 |
| | (PARTICLE SIZE 2.4-2.9 μm) | | | | | | |
| | SYNTHETIC PHLOGOPITE | — | — | — | — | — | — |
| | (PARTICLE SIZE 12.5 μm) | | | | | | |
| | TRIETHOXYSILYLETHYL | — | — | — | — | — | — |
| | POLYDIMETHYLSILOXYETHYL | | | | | | |
| | DIMETHICONE-TREATED | | | | | | |
| | SYNTHETIC PHLOGOPITE | | | | | | |
| | (PARTICLE SIZE 11.3 μm) | | | | | | |
| | SILICA (PARTICLE SIZE 0.55 μm) | — | — | 10.00 | — | — | — |
| | SILICA (PARTICLE SIZE 1 μm) | — | — | — | — | — | — |
| | SILICA (PARTICLE SIZE 5 μm) | — | — | — | — | — | — |
| | CALCIUM MYRISTATE | 5.00 | — | — | — | — | — |
| | (PARTICLE SIZE 7-11 μm) | | | | | | |
| | CELLULOSE | — | 5.00 | — | — | — | — |
| | (PARTICLE SIZE 5-25 μm) | | | | | | |
| LIQUID OIL | DIMETHICONE | — | — | 10.00 | 10.00 | 10.00 | 10.00 |
| | CAPRYLIC/CAPRIC | 11.45 | 11.45 | 22.45 | 32.45 | 22.45 | 22.45 |
| | TRIGLYCERIDE | | | | | | |
| | DISTEARYL MALATE | 10.00 | 10.00 | — | — | — | — |
| | PENTAERYTHRITYL | 13.00 | 13.00 | 8.00 | 8.00 | 8.00 | 8.00 |
| | TETRAETHYLHEXANOATE | | | | | | |
| WAX | PARAFFIN | 10.20 | 10.20 | 10.20 | 10.20 | 10.20 | 10.20 |
| | MICROCRYSTALLINE WAX | 0.8 | 0.80 | 0.80 | 0.80 | 0.80 | 0.80 |
| COLOR MATERIAL | COLOR MATERIAL | 10.00 | 10.00 | 5.00 | 5.00 | 5.00 | 5.00 |
| MOISTURIZER | GLYCERIN | 4.00 | 4.00 | 4.00 | 4.00 | 4.00 | 4.00 |
| | DPG | 6.00 | 6.00 | 6.00 | 6.00 | 6.00 | 6.00 |
| THICKENER | XANTHAN GUM | 0.15 | 0.15 | 0.15 | 0.15 | 0.15 | 0.15 |
| VISCOSITY | SILICA DIMETHYL SILYLATE | 1.00 | 1.00 | — | — | — | — |
| MODIFIER | | | | | | | |
| SURFACTANT | CETYL PEG/PPG-10/ | 4.00 | 4.00 | 4.00 | 4.00 | 4.00 | 4.00 |
| | 1 DIMETHICONE | | | | | | |
| | PEG-60 HYDROGENATED CASTOR OIL | 0.80 | 0.80 | 0.80 | 0.80 | 0.80 | 0.80 |
| STABILIZER | CALCIUM STEARATE | 0.05 | 0.05 | 0.05 | 0.05 | 0.05 | 0.05 |
| PRESERVATIVE | PHENOXYETHANOL | 0.50 | 0.50 | 0.50 | 0.50 | 0.50 | 0.50 |
| AGENT | NIACINAMIDE | 5.00 | 5.00 | — | — | — | — |
| TOTAL (MASS %) | | 100 | 100 | 100 | 100 | 100 | 100 |
| EVALUATION | MATTE FEELING | B | B | D | D | B | B |
| | COMFORT IN APPLICATION | A | A | A | A | A | A |
| | MOISTURIZING FEELING | A | A | A | A | A | A |

TABLE 3

| | | EXAMPLE | EXAMPLE | EXAMPLE | EXAMPLE | EXAMPLE |
|---|---|---|---|---|---|---|
| | | 11 | 12 | 13 | 14 | 15 |
| WATER | WATER | 23.05 | 23.05 | 23.05 | 23.05 | 23.05 |
| POWDER | SYNTHETIC PHLOGOPITE | 5.00 | 5.00 | 5.00 | 5.00 | 5.00 |
| | (PARTICLE SIZE 2.4-2.9 μm) | | | | | |
| LIQUID OIL | DIMETHICONE | — | 10.00 | 20.00 | 30.00 | 35.45 |
| | CAPRYLIC/CAPRIC | 31.45 | 21.45 | 11.45 | 1.45 | — |
| | TRIGLYCERIDE | | | | | |
| | PENTAERYTHRITYL | 8.00 | 8.00 | 8.00 | 8.00 | 4.00 |
| | TETRAETHYLHEXANOATE | | | | | |
| WAX | PARAFFIN | 11.00 | 11.00 | 11.00 | 11.00 | 11.00 |
| COLOR MATERIAL | COLOR MATERIAL | 5.00 | 5.00 | 5.00 | 5.00 | 5.00 |
| MOISTURIZER | GLYCERIN | 4.00 | 4.00 | 4.00 | 4.00 | 4.00 |
| | DPG | 6.00 | 6.00 | 6.00 | 6.00 | 6.00 |
| THICKENER | XANTHAN GUM | 0.15 | 0.15 | 0.15 | 0.15 | 0.15 |
| VISCOSITY | SILICA DIMETHYL SILYLATE | 1.00 | 1.00 | 1.00 | 1.00 | 1.00 |
| MODIFIER | | | | | | |
| SURFACTANT | CETYL PEG/PPG-10/ | 4.00 | 4.00 | 4.00 | 4.00 | 4.00 |
| | 1 DIMETHICONE | | | | | |
| | PEG-60 HYDROGENATED CASTOR OIL | 0.80 | 0.80 | 0.80 | 0.80 | 0.80 |
| STABILIZER | CALCIUM STEARATE | 0.05 | 0.05 | 0.05 | 0.05 | 0.05 |
| PRESERVATIVE | PHENOXYETHANOL | 0.50 | 0.50 | 0.50 | 0.50 | 0.50 |
| TOTAL (MASS %) | | 100 | 100 | 100 | 100 | 100 |
| EVALUATION | MATTE FEELING | B | B | B | A | A |
| | COMFORT IN APPLICATION | A | A | B | C | C |
| | MOISTURIZING FEELING | A | A | B | D | D |

TABLE 4

| | | EXAMPLE | EXAMPLE | EXAMPLE |
|---|---|---|---|---|
| | | 16 | 17 | 18 |
| WATER | WATER | 10.30 | 23.05 | 23.05 |
| POWDER | SYNTHETIC PHLOGOPITE | 13.74 | 5 | 5 |
| LIQUID OIL | DIMETHICONE | 10 | 10 | 10 |
| | CAPRYLIC/CAPRIC | 22.45 | 17.45 | 17.45 |
| | TRIGLYCERIDE | | | |
| | PENTAERYTHRITYL | 8 | 8 | 8 |
| | TETRAETHYLHEXANOATE | | | |
| SEMISOLID | MACADAMIA SEED OIL | — | 5 | — |
| OIL | POLYGLYCERYL-6 | | | |
| | ESTERS BEHENATE | | | |
| FILM- | TRIMETHYLSILOXYSILICATE | — | — | 3 |
| FORMING | | | | |
| AGENTS | ISODODECANE | — | — | 2 |
| WAX | PARAFFIN | 11 | 11 | 11 |
| COLOR MATERIAL | COLOR MATERIAL | 5 | 5 | 5 |
| MOISTURIZER | GLYCERIN | 3.43 | 4 | 4 |
| | DPG | 11 | 6 | 6 |
| THICKENER | XANTHAN GUM | — | 0.15 | 0.15 |
| SURFACTANT | CETYL PEG/PPG-10/ | 4 | 4 | 4 |
| | 1 DIMETHICONE | | | |
| | PEG-60 HYDROGENATED CASTOR OIL | 0.69 | 0.8 | 0.8 |
| STABILIZER | CALCIUM STEARATE | 0.05 | 0.05 | 0.05 |
| PRESERVATIVE | PHENOXYETHANOL | 0.34 | 0.5 | 0.5 |
| TOTAL (MASS %) | | 100 | 100 | 100 |
| EVALUATION | MATTE FEELING | B | B | B |
| | COMFORT IN APPLICATION | A | A | A |
| | MOISTURIZING FEELING | A | A | A |

As described in Tables 1 to 4, the water-in-oil emulsified solid cosmetics of Examples 1 to 18 exhibited good results in matte feeling, comfort in application, and moisturizing feeling. In contrast, the water-in-oil emulsified solid cosmetic of Comparative Example 1 in which the particle size of the powder was less than 1.0 μm, and the water-in-oil emulsified solid cosmetic of Comparative Example 2 in which powder was not contained, gave a glossy finish, and the matte feeling was remarkably poor.

Although the embodiments have been described above, the above embodiments are presented as examples, and the present invention is not limited by the above embodiments. The above embodiments can be implemented in various other forms, and various combinations, omissions, substitutions, changes, and the like can be made without departing from the scope of the invention. These embodiments and variations thereof are included in the scope and gist of the invention, and are also included in the inventions described in the claims and their equivalent scope.

The present application claims priority to Japanese Patent Application No. 2022-113474 filed Jul. 14, 2022, the entire contents of which are incorporated herein by reference.

1. A water-in-oil emulsified solid cosmetic comprising:
an internal aqueous phase in which powder is dispersed in an aqueous medium; and
an external oil phase thickened or solidified with wax, wherein
an average primary particle size of the powder is 1.0 μm or more.

2. The water-in-oil emulsified solid cosmetic according to claim 1, wherein the powder contains at least one of mica, silica, silicone, talc, cellulose powder, or lauroyl lysine.

3. The water-in-oil emulsified solid cosmetic according to claim 1, wherein the powder is inorganic powder.

4. The water-in-oil emulsified solid cosmetic according to claim 1, wherein the external oil phase contains a color material.

5. The water-in-oil emulsified solid cosmetic according to claim 4, wherein the color material includes an organic pigment or an oil-soluble dye.

6. The water-in-oil emulsified solid cosmetic according to claim 1, wherein the external oil phase contains 15 mass % or more of residual oil with respect to the water-in-oil emulsified solid cosmetic.

7. The water-in-oil emulsified solid cosmetic according to claim 1, wherein the external oil phase contains 15 mass % or less of semisolid oil with respect to the water-in-oil emulsified solid cosmetic.

8. The water-in-oil emulsified solid cosmetic according to claim 1, wherein the external oil phase contains 20 mass % or less of a film-forming agent with respect to the water-in-oil emulsified solid cosmetic.

9. The water-in-oil emulsified solid cosmetic according to claim 1, wherein the water-in-oil emulsified solid cosmetic is a lipstick.
